(19) United States
(12) Patent Application Publication
(10) Pub. No.: US 2004/0158103 A1
(43) Pub. Date: Aug. 12, 2004

(54) Cyclohexane oxidation catalysts

(75) Inventors: Larisa V. Pirutko, Novosibirsk (RU); Alexander S. Kharitonov, Novosibirsk (RU); Mikhail I. Khramov, Pensacola, FL (US); Anthony K. Uriarte, Pensacola, FL (US)

Correspondence Address: CRAIG M. LUNDELL, HOWREY SIMON ARNOLD & WHITE, LLP, 750 BERING DRIVE, HOUSTON, TX, 77057, US

(73) Assignee: Solutia Inc., St. Louis, MO

(21) Appl. No.: 10/361,608

(22) Filed: Feb. 11, 2003

(51) Int. Cl. C07C 27/10

(52) U.S. Cl. 568/360

(57) ABSTRACT

A cycloalkane, such as cyclohexane, is catalytically oxidized to form a ketone/alcohol mixture. A cycloalkane-containing reaction mixture is contacted with a source of oxygen in the presence of a catalytic effective amount of gold supported on a crystalline zeolite-like support. The zeolite-like support optionally contains one or more heteroatoms.

### FIELD OF THE INVENTION

The present invention is directed to catalytic oxidation of cycloalkanes to form mixtures containing the corresponding ketones and alcohols.

### BACKGROUND OF THE INVENTION

Several different processes have been used for the oxidation of cyclohexane into a product mixture containing cyclohexanone and cyclohexanol. Such product mixture is commonly referred to as a KA (ketone/alcohol) mixture. The KA mixture can be readily oxidized to produce adipic acid, which is an important reactant in processes for preparing certain condensation polymers, notably polyamides. Given the large quantities of adipic acid consumed in these and other processes, there is a need for cost-effective processes for producing adipic acid and its precursors.

One technique presently used for cyclohexane oxidation employs metaboric acid as a catalyst. Although metaboric acid is a somewhat effective oxidation catalyst, certain drawbacks are associated with its use. A principal drawback is the need for catalyst recovery, which typically involves hydrolysis of the reaction mixture, aqueous and organic phase separation, and dehydration of boric acid. These steps introduce considerable complexity and expense into the overall process.

Organic cobalt salts, such as cobalt octanoate, have been widely used for oxidizing cyclohexane into KA mixtures. Various homogenous metal catalysts also have been proposed for oxidizing cycloalkanes, such as salts of chromium, iron, and manganese, with varying results in terms of cyclohexane conversion and ketone/alcohol selectivities.

Two-stage processes also have been used for cycloalkane oxidation. In a first stage of one typical two-stage process, cyclohexane is oxidized to form a reaction mixture containing cyclohexyl hydroperoxide (CHHP). In a second stage, CHHP is decomposed, with or without use of a catalyst, to form a KA mixture. An example of a two-stage process is described in U.S. Pat. No. 6,284,927 to Druliner et al., in which an alkyl or aromatic hydroperoxide is oxidized in the presence of a heterogeneous catalyst of Au, Ag, Cu or a sol-gel compound containing particular combinations of Fe, Ni, Cr, Co, Zr, Ta, Si, Mg, Nb, Al and Ti, wherein certain of these metals are combined with an oxide. Other catalysts that have been proposed for the second stage of two-stage oxidation processes include salts of manganese, iron, cobalt, nickel, and copper.

WO 00/53550 and companion U.S. Pat. No. 6,160,183 to Druliner et al. describe a heterogeneous catalyst for so-called direct oxidation of cycloalkanes to form a KA mixture. The catalysts described include gold, gold sol-gel compounds, and sol-gel compounds containing particular combinations of Cr, Co, Zr, Ta, Si, Mg, Nb, Al and Ti, wherein certain of these metals are combined with an oxide.

Fan et al., “Environmentally Benign Oxidations of Cyclohexane and Alkenes with Air Over Zeolite-encapsulated Au Catalysts,” Lanzhou Institute of Chemical Physics, Chinese Academy of Sciences, discloses catalysts for the oxidation of cyclohexane and alkenes. Au/NaY is said to yield high turnover frequency and product selectivities when used as an oxidation catalyst for cyclohexane.

There remains a need for cost-effective methods for oxidizing cycloalkanes to KA mixtures, particularly methods employing catalysts that yield high cycloalkane conversions, high ketone and alcohol selectivities, and relatively low cycloalkyl hydroperoxide concentrations.

### SUMMARY OF THE INVENTION

The present invention is directed to a method of oxidizing a cycloalkane in a reaction mixture to form a product mixture containing a corresponding alcohol and ketone. The method comprises contacting the reaction mixture with a source of oxygen in the presence of a catalytic effective amount of gold supported on a zeolite-like support. Crystalline silicates iso-structural with zeolites and crystal phosphates iso-structural with zeolites may be used as a zeolite-like support for preparing the gold-containing catalysts.

Crystalline silicates optionally may contain one or more heteroatoms and can be described by the general formula: (El₂Oₙ)ₓSiO₂, where x≦0.13, El is at least one element of Periods 2, 3, 4, and 5 of the periodic system, and n is valence of the element El.

Crystalline phosphates optionally may contain one or more heteroatoms and can be described by the general formula: (El₂Oₙ₎ₓ(Al₂O₃)_{y}P₂O₅, where x≦0.27, y≦1.0, El is at least one element of Periods 2, 3, 4, and 5 of the periodic system, and n is valence of the element El.

The supported gold-containing catalysts of the present invention have been found to yield product mixtures characterized by high cycloalkane conversions, high ketone and alcohol selectivities, and relatively low cycloalkyl hydroperoxide concentrations. These catalysts thus exhibit exceptional performance in cycloalkane oxidation. In addition, the insoluble heterogeneous catalyst provides a significantly simplified operation compared to the use of boric acid as catalyst. For example, catalyst recovery may be unnecessary if a catalyst basket or the like is used. The insoluble heterogeneous catalyst also can be used as a slurry and easily recovered by filtration or centrifugation.

### DETAILED DESCRIPTION OF THE INVENTION

The present invention is directed to methods for catalytically oxidizing cycloalkanes. The term “cycloalkane,” as used herein, refers to saturated cyclic hydrocarbons having from 3 to about 10 carbon atoms, more usually from about 5 to about 8 carbon atoms. Non-limiting examples of cycloalkanes include cyclopentane, cyclohexane, cycloheptane, and cyclooctane.

The catalyst of the present invention comprises gold supported on a zeolite-like support. Gold can be supplied in any suitable form. For example, it can be deposited onto the support by impregnation, precipitation, deposition-precipitation, ion-exchange, anion or cation adsorption from solutions, or vapor phase deposition. In addition, gold-containing catalysts can be prepared by introducing the source of gold at the stage of hydrothermal synthesis of the support material. When using the above-mentioned and other possible methods, the amount of gold introduced can vary over a wide range but usually is up to about 10 wt %. The catalyst typically contains ultra-fine sized gold particles, e.g., from about 3 to 15 nm in diameter.

A zeolite-like crystalline silicate support can have a variety of structures, non-limiting examples of which include BEA, FAU, FER, MFI, MEL, MOR, MTW, MTT, MCM-22, MCM-41, MCM-48, and NU-1. A fraction of silicon in the crystalline silicate may be isomorphly or non-isomorphly replaced by one or more heteroatoms selected from the group consisting of B, Be, Al, Ga, In, Ge, Sn, Ti, Zr, Hf, V, Cr, Mn, Fe, Co, P, Mo, and W. If the replacement is isomorphous and the valence of the replacing element is not equal to the valence of silicon, a corresponding crystalline silicate may contain in cationic positions hydrogen cations, and/or cations of alkaline (e.g., Li⁺, Na⁺, K⁺, etc.) and/or alkaline-earth metal (Mg²⁺, Ca²⁺, Sr²⁺, etc.) and/or cations and/or oxy-cations of any transitional metal (e.g., Cu⁺, Zn²⁺, AlO⁺, VO⁺, FeO⁺, etc.).

A zeolite-like phosphate-based support also can be used for preparation of gold-containing catalysts, which provide optimal catalyst performance in cycloalkane oxidation. The phosphate-based, porous support can have a variety of structures, non-limiting examples of which include AFI, AEL, AFO, AFR, AFS, AFT, AFY, ATN, ATO, ATS, ATT, ATV, and AWW. A fraction of phosphorous in the crystalline phosphate can be isomorphly or non-isomorphly replaced by one or more heteroatoms selected from the group consisting of Ai, Si, B, Be, Ga, In, Ge, Sn, Ti, Zr, Hf, V, Cr, Mn, Fe, Co, Mo, and W.

Gold-containing catalysts on zeolite-like supports, such as Au/TS-1, have been found to provide exceptionally good performance, particularly high ketone/alcohol selectivities at relatively high cyclohexane conversions. Preferred catalysts of the present invention provide selectivities of about 90% at cyclohexane conversions up to about 6-7%. In the absence of air, the catalyst selectively decomposes cyclohexyl hydroperoxide (CHHP) to cyclohexanol and cyclohexanone.

Crystalline structure of zeolite-like material is formed by tetrahedral fragments (e.g., [SiO₄]⁴⁻, [AlO₄]⁵⁻, [PO₄]³⁺) combined by their common vertices into a three-dimensional framework with cavities and channels. The above-described methods for preparing gold-containing catalysts provide arrangement of ultra-fine sized gold particles in the channels and cavities of the micropore space of a zeolite-like support, or in the pore entrances of the external surface of zeolite crystals. Such arrangement of gold particles prevents sintering during thermal treatment of the catalyst and increases operation stability.

Zeolite-like supports can be prepared in accordance with methods well known to persons skilled in the art (J. Weitkamp, L. Puppe (Eds), Catalysis and Zeolites: Fundamentals and Applications, Springer, pp. 1-52). For example, crystalline silicates can be prepared by the following method. A mixture containing a source of silicon, a source of Elⁿ⁺ (if needed), an alkali, organic surfactants and, in some cases, a crystallization seed, is homogenized and then placed into an autoclave. The mixture is kept under hydrothermal conditions for 10 hours to 30 days at a temperature within a range of from 80° C. to 200° C. Prior to use as a support, the solid product may be calcined at a temperature ranging from 450° C. to 800° C. A zeolite-like silicate of desired structure may be produced by varying the chemical composition of the mixture as well as the temperature and time of hydrothermal synthesis.

In addition, the gold-containing catalyst may be subjected to post-synthesis treatment including, but not limited to, washing with acids or chelating agents, treatment with a reducing or oxidizing or inert gas, or mixtures of steam with reducing or oxidizing or inert gas.

Zeolite-like crystalline phosphates can be prepared using any known method (J. Weitkamp, L. Puppe (Eds), Catalysis and Zeolites: Fundamentals and Applications, Springer, pp. 53-80). A mixture containing a source of phosphorous, a source of aluminum and/or Elⁿ⁺ (if needed), an alkali, organic surfactants and, in some cases, a crystallization seed, is homogenized and then placed into an autoclave. The mixture is kept under hydrothermal conditions for 10 hours to 30 days at a temperature within a range of from 80° C. to 200° C. Prior to use as a support, the solid product may be calcined at a temperature ranging from 450° C. to 800° C. for the removal of organic inclusions. A crystalline phosphate of given structure may be produced by varying the source of phosphorous and aluminum, the nature of the organic surfactant, and the temperature and time of hydrothermal synthesis.

In the practice of the invention, the catalysts can be contacted with a cycloalkane, such as cyclohexane, by formulation into a catalyst bed, which is arranged to provide intimate contact between the catalyst and reactants. Alternatively, catalysts can be slurried with reaction mixtures using techniques known in the art. The process of the invention is suitable for either batch or continuous cycloalkane oxidation. These processes can be performed under a wide variety of conditions, as will be apparent to persons of ordinary skill.

Suitable reaction temperatures for the process of the invention typically range from about 130 to about 200° C., more usually from about 150 to about 180° C. Reaction pressures often range from about 69 kPa to about 2760 kPa (10-400 psi), more usually from about 276 kPa to about 1380 kPa (40-200 psi) in order to keep the reaction mixture in the liquid phase. Cycloalkane reactor residence time generally varies in inverse relation to reaction temperature, and typically is less than about 120 minutes.

The source of oxygen used in the oxidation may be molecular oxygen itself but is conveniently air or other mixtures of nitrogen and oxygen with a higher or lower proportion of oxygen than that of air, obtained, for example, by mixing oxygen or nitrogen with air. However, air is preferred. The time of contacting air with the liquid phase in the reactor usually varies from about 0.05 to 5 min., more usually from about 0.2 to about 1.5 min.

The following examples are provided for illustrative purposes only and should not be regarded as limiting the invention.

### EXAMPLE 1

This example illustrates preparing a gold-containing catalyst using a silicalite-1 as a support, a zeolite-like material with MFI structure. Silicalite-1 (3 g) was suspended in 50 ml water, and 7.9 ml of 0.05 M HAuCl₄ was added dropwise to this suspension. The pH of the resulting slurry was adjusted to 7 with 6% aqueous ammonia, and the slurry was agitated for an additional 2 hours. The precipitate was filtered, dried 10 hours at 100° C., and calcined 2 hours at 200° C.

### Preparation of Silicalite-1 Support

Silicalite-1 was prepared as described in Flanigen E. M., Bennett J. M, Grose R. W., Cohen J. P., Patton R. L., Kirchner R. M., Smith J. V. (1978) Nature 271, 512. Silica sol (36 g) containing 40% SiO₂ was added to 220 ml 0.1 M tetrapropylammonium hydroxide solution. The mixture was agitated for 30 min., and 4 ml of 10M NaOH aqueous solution was added to it dropwise. The resulting mixture was agitated for 1 hr. at ambient temperature and kept for 72 hr. at 170° C. The precipitate was filtered, washed with distilled water, and dried for 24 hrs. at 100° C. X-ray diffraction analysis confirmed the MFI structure of the obtained product.

### EXAMPLE 2

This example illustrates preparing a gold-containing catalyst using a titanosilicate TS-1 of MFI structure having the following composition: (TiO₂)_{0.025}(SiO₂). The catalyst was prepared according to Example 1, wherein the crystalline titanium silicate TS-1 was used as support.

### Preparation of TS-1 Support

Tetraethylorthosilicate (91 g) was placed in a glass flask under inert atmosphere.

Tetraethyltitanate (3 g) was added under stirring. Tetrapropylammonium hydroxide (25% aqueous solution, 160 g) was added and the mixture was stirred 1 hr. at ambient temperature and 5 hr. at 80-90° C. After that time, the mixture was transferred into an autoclave and kept 10 days at 175° C. The product was cooled, filtered, and washed with distilled water. Organic template was removed by calcinations for 3 hr. at 550° C. X-ray diffraction analysis showed that the product had MFI structure.

### EXAMPLE 3

This example illustrates preparing a gold-containing catalyst using a borosilicate of MFI structure having the following composition: (B₂O₃)_{0.0083}(SiO₂). The catalyst was prepared according to Example 1, wherein borosilicate was used as support.

### Preparation of Borosilicate Support

Tetrapropylammonium hydroxide (25% aqueous solution, 61 g) was placed in a glass flask under an inert atmosphere. Boric acid (9.3 g) was added under stirring. Tetraethylorthosilicate (93.75 g) was added and the mixture was gradually heated to 60° C. and kept at this temperature under constant stirring for 12 hours. After that time, KOH (0.09 g) and distilled water were added to make the overall volume 150 ml. The mixture was transferred into an autoclave and kept for 12 days at 145° C. The product was cooled, filtered, washed with distilled water, and dried at 120° C. Organic template was removed by calcination at 750° C. X-ray diffraction analysis showed that the product had MFI structure. The material is referred to as B-ZSM-5 below.

### EXAMPLE 4

This example illustrates preparing a gold-containing catalyst on an alumosilicate of MFI structure having the following composition: (Al₂O₃)_{0.00025}(SiO₂). The catalyst was prepared according to Example 1, wherein alumosilicate was used as support. The support was prepared as described in Argauer et al., U.S. Pat. No. 3,702,886, the disclosure of which is hereby incorporated by reference.

### EXAMPLE 5

This example illustrates preparing a gold-containing catalyst using an alumosilicate of MFI structure having the following composition: (Al₂O₃)_{0.086}(SiO₂). The catalyst was prepared according to Example 1, wherein alumosilicate was used as support. The support was prepared as described in Argauer et al., U.S. Pat. No. 3,702,886, and was additionally treated with steam at 650° C. for 2 hrs. prior to gold deposition.

### EXAMPLE 6

This example illustrates preparing a gold-containing catalyst on an alumosilicate of FAU structure having the composition: (Na₂O)_{0.026}(Al₂O₃)_{0.15}(SiO₂). The catalyst was prepared according to Example 1, wherein 0.03 M aqueous HAuCl₄ solution was used as a source of gold. The support was prepared as described in Breck, U.S. Pat. No. 3,130,007, the disclosure of which is hereby incorporated by reference.

### EXAMPLE 7

This example illustrates preparing a gold-containing catalyst on an alumosilicate of FAU structure having the composition: (Na₂O)_{0.0O25}(Al₂O₃)_{0.023}(SiO₂). The catalyst was prepared according to Example 1, wherein 0.03 M aqueous HAuCl₄ solution was used as a source of gold. The support was prepared as described in Kerr G. T., JPhys. Chem. 71 (1967) 4155.

### EXAMPLE 8

This example illustrates preparing a gold-containing catalyst on a crystalline alumophosphate with ATS structure having the composition: Al₂O₃•P₂O₅. The catalyst was prepared according to Example 1, wherein alumophosphate was used as support. The support was prepared as described in Bennet J. M., Richardson J. M., Pluth J. J., Smith J. V. (1987), Zeolites 7, 160.

### EXAMPLES 9-23

Examples 9-23 illustrate using the catalysts of Examples 1-7 for the oxidation of cyclohexane.

Each of the catalysts according to Examples 1-7 (0.8-0.9 g) was loaded into 300 ml Parr pressure reactor containing cyclohexane (160 g) and cyclohexanone (0 or 0.70 g). The reactor was purged for 20 minutes with helium (300 cc/min at atmospheric pressure) and, after that, pressurized with helium to 130-140 psig.

The contents of the reactor were heated to 150° C. or 170° C., helium flow was shut down, and air was fed to the reactor at the rate of 300 cc/min. until desired cyclohexane conversion was achieved. Results of the tests are indicated in Table 1.

### COMPARATIVE EXAMPLES 24-32

Comparative examples 24-32 illustrate oxidizing cyclohexane without a catalyst. Cyclohexane (160 g) and cyclohexanone (0 or 0.70 g) were loaded into 300 ml Parr pressure reactor. The reactor was purged for 20 minutes with 300 cc/min. helium at atmospheric pressure and, after that, pressurized with helium to 130-140 psig. The contents of the reactor were heated to 150° C. or 170° C., helium flow was shut down, and air was fed to the reactor at the rate of 300 cc/min. until desired cyclohexane conversion was achieved. Results of the tests are indicated in Table 1.

### COMPARATIVE EXAMPLE 33

This comparative example illustrates oxidizing cyclohexane using borosilicate B-ZSM-5 with MFI structure as a catalyst (without gold).

Boron-containing ZSM-5 (B-ZSM-5) was prepared according to the first part of Example 3 (the catalyst did not contain gold). The catalyst was tested in cyclohexane oxidation as in Examples 8-23. Results of the test are shown in Table 1.

### COMPARATIVE EXAMPLE 34

This comparative example illustrates oxidizing cyclohexane using silicalite-1 with MFI structure as a catalyst (without gold).

Silicalite-1 was prepared according to Example 1 (the catalyst did not contain gold). The catalyst was tested in cyclohexane oxidation as in Examples 8-23. Results of the test are shown in Table 1.

### COMPARATIVE EXAMPLE 35

This comparative example illustrates oxidizing cyclohexane using titanosilicate TS-1 with MFI structure as a catalyst (without gold).

Titanosilicate TS-1 was prepared according to the first part of Example 2 (the catalyst did not contain gold). The catalyst was tested in cyclohexane oxidation as in Examples 8-23. Results of the test are shown in Table 1.

### COMPARATIVE EXAMPLE 36

This comparative example illustrates oxidizing cyclohexane using crystalline alumophosphate as a catalyst (without gold).

Crystalline alumophosphate was prepared according to the first part of Example 7 (the catalyst did not contain gold). The catalyst was tested in cyclohexane oxidation as in Examples 8-23. Results of the test are shown in Table 1.

### COMPARATIVE EXAMPLE 37

This comparative example illustrates preparing an Au/SiO₂ catalyst having an amorphous structure, and using the catalyst in cyclohexane oxidation.

Au/SiO₂ catalyst was prepared using a sol-gel method. Tetraethylorthosilicate (10.5 g) and 1.3 ml of 0.12 M HAuCl₄ solution were dissolved in ethanol, and dilute aqueous NH₃ solution was added until the mixture became turbid. The mixture was held for 15 hours, and then the precipitate was filtered, washed, and dried. The catalyst was tested in cyclohexane oxidation as in Examples 8-23. Results of the tests are indicated in Table 1.

### COMPARATIVE EXAMPLE 38

This comparative example illustrates preparing an Au/Al₂O₃ catalyst having a boehmite structure, and using the catalyst in cyclohexane oxidation.

y-Al₂O₃ (basic, Alpha Aesar, 5.984 g) was suspended in 117.3 g solution of 0.1% AuCl₃ in 0.5% HCl. The slurry was titrated with 9% NH₃ to pH 7.0. The agitation continued for 4 hours at ambient temperature. The slurry was filtered, and the precipitate was washed on a filter with 50 ml water, dried overnight at 110° C., and calcined 3 hours at 450° C. The catalyst, Au/Al20₃ was tested in cyclohexane oxidation as in Examples 8-23. Results of the test are indicated in Table 1.

TABLE 1

| | Catalyst (amount of Au | | | Cyclohexanone | Cyclohexane | Selectivity to | Product | | |
|---|---|---|---|---|---|---|---|---|---|
| | is indicated as molar | Support | T | in reactor | conversion | K + A + CHP | distribution (%) | | |
| Ex. | fraction) | Structure | (° C.) | charge (g) | (%) | (%) | K | A | CHP |
| 9 | 4.6 · 10⁻³ Au on SiO₂ | MFI | 170 | 0.7 | 3.39 | 91.4 | 52 | 45 | 4 |
| 10 | 4.6 · 10⁻³ Au on SiO₂ | MFI | 150 | 0 | 2.93 | 95.9 | 17.3 | 34.3 | 49.4 |
| 11 | 4.6 · 10⁻³ Au on SiO₂ | MFI | 150 | 0 | 6.09 | 88 | 22.4 | 43.4 | 34.2 |
| 12 | 5.7 · 10⁻³ Au on | MFI | 170 | 0.7 | 3.56 | 90 | 52 | 42 | 6 |
| | (Al₂O₃)_{0.0025}SiO₂ | | | | | | | | |
| 13 | 5.0 · 10⁻³ Au on | MFI | 170 | 0.7 | 3.83 | 84 | 35.3 | 53.7 | 11 |
| | (Al₂O₃)_{0.086}SiO₂ | | | | | | | | |
| 14 | 5.7 · 10⁻³ Au on | MFI | 170 | 0.7 | 3.2 | 92 | 50 | 47 | 3 |
| | (TiO₂)_{0.025}(SiO₂) | | | | | | | | |
| 15 | 5.7 · 10⁻³ Au on | MFI | 170 | 0.7 | 4.21 | 90.6 | 50 | 47 | 4 |
| | (TiO₂)_{0.025}(SiO₂) | | | | | | | | |
| 16 | 5.7 · 10⁻³ Au on | MFI | 170 | 0.7 | 6.66 | 88.1 | 53 | 47 | 0 |
| | (TiO₂)_{0.025}(SiO₂) | | | | | | | | |
| 17 | 5.7 · 10⁻³ Au on | MFI | 150 | 0 | 4.11 | 91.2 | 28.5 | 52.4 | 19.1 |
| | (TiO₂)_{0.025}(SiO₂) | | | | | | | | |
| 18 | 5.3 · 10⁻³ Au on | MFI | 170 | 0.7 | 2.53 | 90.3 | 38 | 45 | 16 |
| | (B₂O₃)_{0.0083}(SiO₂) | | | | | | | | |
| 19 | 5.3 · 10⁻³ Au on | MFI | 170 | 0.7 | 3.11 | 86.6 | 34 | 51 | 15 |
| | (B₂O₃)_{0.0083}(SiO₂) | | | | | | | | |
| 20 | 5.3 · 10⁻³ Au on | MFI | 170 | 0.7 | 4.88 | 87.9 | 46 | 48 | 6 |
| | (B₂O₃)_{0.0083}(SiO₂) | | | | | | | | |
| 21 | 2 · 10⁻² Au on | FAU | 170 | 0.7 | 3.98 | 83.7 | 27 | 55 | 18 |
| | (Na₂O)_{0.026}(Al₂O₃)_{0.15} | | | | | | | | |
| | (SiO₂) | | | | | | | | |
| 22 | 3.2 · 10⁻³ Au on | FAU | 170 | 0.7 | 2.6 | 85 | 32.2 | 62.5 | 5.3 |
| | (Na₂O)_{0.0025}(Al₂O₃)_{0.023} | | | | | | | | |
| | (SiO₂) | | | | | | | | |
| 23 | 2.4 · 10⁻² Au on | ATS | 170 | 0.7 | 2.9 | 87 | 28 | 55 | 17 |
| | Al₂O₃.P₂O₅ | | | | | | | | |
| 24 | None | — | 170 | 0 | 1.63 | 87.3 | 7 | 11 | 8 |
| 25 | None | — | 170 | 0 | 4.77 | 87.9 | 14 | 27 | 59 |
| 26 | None | — | 170 | 0 | 7.35 | 81.5 | 20 | 44 | 36 |
| 27 | None | — | 170 | 0.7 | 2.76 | 87.5 | 14 | 29 | 57 |
| 28 | None | — | 170 | 0.7 | 3.68 | 84.5 | 16 | 41 | 43 |
| 29 | None | — | 170 | 0.7 | 4.94 | 83 | 19 | 55 | 26 |
| 30 | None | — | 170 | 0.7 | 6.32 | 80.2 | 23 | 57 | 21 |
| 31 | None | — | 150 | 0 | 3.56 | 96.6 | 8.2 | 14.9 | 77 |
| 32 | None | — | 150 | 0 | 8.45 | 88.4 | 14.8 | 27.1 | 58.1 |
| 33 | (B₂O₃)_{0.0083}(SiO₂) | MFI | 170 | 0.7 | 2.78 | 87.3 | 15 | 36 | 49 |
| 34 | SiO₂ | MFI | 170 | 0.7 | 3.7 | 88 | 8 | 44 | 48 |
| 35 | (TiO₂)_{0.025}(SiO₂) | MFI | 170 | 0.7 | 3.7 | 86 | 11 | 43 | 46 |
| 36 | Al₂O₃.P₂O₅ | ATS | 170 | 0.7 | 3.2 | 85.4 | 10 | 43 | 48 |
| 37 | 4.5 · 10⁻³ Au on SiO₂ | amorphous | 170 | 0.7 | 3.19 | 89.4 | 19 | 32 | 48 |
| 38 | 4.3 · 10⁻³ Au on Al₂O₃ | boehmite | 170 | 0.7 | 2.56 | 87.7 | 7 | 31 | 62 |

It will be understood that while the invention has been described in conjunction with specific embodiments thereof, the foregoing description and examples are intended to illustrate, but not limit the scope of the invention. Other aspects, advantages and modifications will be apparent to those skilled in the art to which the invention pertains, and these aspects and modifications are within the scope of the invention, which is limited only by the appended claims.

What is claimed is:

1. A method of oxidizing a cycloalkane in a reaction mixture to form a product mixture containing a corresponding alcohol and ketone, the method comprising contacting the reaction mixture with a source of oxygen in the presence of a catalytic effective amount of a gold-containing catalyst supported on a crystalline zeolite-like support.

2. The method of claim 1 wherein the cycloalkane is cyclohexane.

3. The method of claim 1 wherein the zeolite-like support is elemento-silicate having a structure selected from the group consisting of BEA, FAU, FER, MFI, MEL, MOR, MTW, MTT, MCM-22, MCM-41, MCM-48, and NU-1.

4. The method of claim 1 wherein the zeolite-like support is elemento-phosphate having a structure selected from the group consisting of AFI, AEL, AFO, AFR, AFS, AFT, AFY, ATN, ATO, ATS, ATT, ATV, and AWW.

5. The method of claim 1 wherein the zeolite-like support comprises a heteroatom.

6. The method of claim 5 wherein said heteroatom is selected from the group consisting of the element of Periods 2, 3, 4, and 5, and mixtures thereof.

7. The method of claim 3 wherein the crystalline silicate support has an MFI structure.

8. The method of claim 7 wherein the silicate support is titanosilicalite.

9. The method of claim 7 wherein the silicate support is borosilicalite.

10. The method of claim 3 wherein the crystalline silicate support has an FAU structure.

11. The method of claim 4 wherein the crystalline phosphate support is an alumophosphate having an ATS structure.

12. The method of claim 4 wherein the crystalline phosphate support is an alumophosphate having an AFI structure.
